(19) United States
(12) Patent Application Publication
(10) Pub. No.: US 2025/0235664 A1
(43) Pub. Date: Jul. 24, 2025

(54) DEVICES, SYSTEMS AND METHODS FOR DELIVERING FLUIDS

(71) Applicants: BOSTON SCIENTIFIC SCIMED, INC., Maple Grove, MN (US); BOSTON SCIENTIFIC MEDICAL DEVICE LIMITED, Galway (IE)

(72) Inventors: Sharath Kumar G., Kanakapura (IN); Deepak Kumar Sharma, Muzaffarnagar (IN); Kevin L. Bagley, Natick, MA (US)

(73) Assignees: BOSTON SCIENTIFIC SCIMED, INC., Maple Grove, MN (US); BOSTON SCIENTIFIC MEDICAL DEVICE LIMITED, Galway (IE)

(21) Appl. No.: 19/033,085

(22) Filed: Jan. 21, 2025

Related U.S. Application Data
(60) Provisional application No. 63/623,582, filed on Jan. 22, 2024.

(51) Int. Cl. A61M 25/00

(52) U.S. Cl. CPC A61M 25/0074; A61M 25/0069; A61M 25/0071; A61M 2025/0004; A61M 2025/0073; A61M 2202/0007; A61M 2202/04; A61M 2205/3331

(57) ABSTRACT

A fluid delivery system having a fluid delivery device configured to deliver a fluid in a spray form. The fluid may be a first fluid and a second fluid mixed together in a manner causing the first fluid to be delivered in a spray form. The first fluid may be a viscous fluid, such as a hemostatic agent, and the second fluid may be a pressurized fluid. The second fluid may be directed at the first fluid to cause the first fluid to be delivered in a spray form. The fluid delivery device may be adjustable to adjust the spray form. The lumens through the fluid delivery device and through the fluid delivery system are formed by channel walls and are uninterrupted by additional/separate components (e.g., valves, etc.) therein affecting fluid flow through the lumens.

### CROSS-REFERENCE TO RELATED APPLICATIONS

This application claims the benefit of priority under 35 U.S.C. § 119 to U.S. Provisional Application No. 63/623,582, filed Jan. 22, 2024, the entire disclosure of which is hereby incorporated by reference herein for all purposes.

### FIELD

The present disclosure relates generally to the field of devices, systems, and methods for delivering fluids to a patient. More particularly, the present disclosure relates to devices, systems, and methods for delivering fluids in a spray form to a patient. Even more particularly, at least one of the fluids may be a viscous fluid, such as a hemostatic fluid. Alternatively or additionally, the devices, systems, and methods may be configured for delivery of fluids within a patient's body.

### BACKGROUND

Various medical procedures involve delivery of a material (e.g., a fluid such as a liquid, such as water or saline, or a gas such as air, carbon dioxide, etc.; or a solid such as a powder or film; etc.) to a patient. For instance, medical procedures within a patient's body may require simple, rapid, and effective hemostasis which may be achieved by delivering a hemostatic material. The hemostatic material may be delivered as a powder or in fluid form. Typically, a fluid form of a hemostatic material is viscous. There has been a growing interest in minimally-invasive, e.g., transluminal, transcatheter, endoscopic, etc., procedures which do not require open surgery (cutting open the patient), but, instead, access a target or treatment site within the patient via a natural orifice and/or body passage (or, in some instances, a small incision not considered to constitute an open-surgery cut). As may be appreciated, delivery of a material into a patient's body using a minimally-invasive approach typically requires the use of tubular members small enough to navigate within a natural body passage. And, as may be further appreciated, delivery of viscous fluids through a lumen of a tubular member capable of delivering a material into a patient's body presents various challenges. The viscosity of the fluid generally requires increased pressure over pressure required to deliver less viscous or non-viscous fluids. Moreover, if delivery of a spray is desired, a greater force is required to propel the fluid than would be required simply to deliver the fluid in non-spray form. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a fluid delivery device includes a proximal end; a distal end; a first lumen defined through the fluid delivery device to deliver a first fluid to the distal end of the fluid delivery device; and a second lumen defined through the fluid delivery device to deliver a second fluid to the distal end of the fluid delivery device. In some aspects, the first lumen and second lumen end and open to a distal delivery end of the fluid delivery device to deliver the first and second fluids to the distal delivery end and to cause the first and second fluids to be mixed and to be delivered in a spray form.

In some aspects, the first lumen and second lumen are concentrically arranged with respect to each other. In some aspects, the proximal end is configured to be fluidly coupled with a tubular elongate member defining a first lumen concentrically positioned within a second lumen, the first lumen of the fluid delivery device is configured and positioned to be fluidly coupled with the first lumen of the tubular elongate member, and the second lumen of the fluid delivery device is configured and positioned to be fluidly coupled with the second lumen of the tubular elongate member.

In some aspects, the fluid delivery device comprises an inner fluid delivery device body defining the first lumen longitudinally therethrough and having an exterior surface with one or more grooves therein defining the second lumen. In some aspects, the distal delivery end of the fluid delivery device defines one or more channels therethrough, the one or more grooves defining the second lumen fluidly coupled with respective ones of the one or more channels to define the second lumen. In some aspects, the one or more channels end and open to a proximally inwardly tapered distal end of the distal delivery end to direct the second fluid toward the first fluid as the first fluid exits the first lumen to deliver the first and second fluids in a spray form. In some aspects, the fluid delivery device further includes an outer fluid delivery device body defining an interior, wherein the inner fluid delivery device body is positioned within the interior of the outer fluid delivery device body, the interior of the outer fluid delivery device body defining the second lumen with the one or more grooves in the exterior surface of the inner fluid delivery device body. In some aspects, the inner fluid delivery device body and the outer fluid delivery device body are movable with respect to each other to adjust the flow of fluid through the distal delivery end of the fluid delivery device. In some aspects, the inner fluid delivery device body has a distal end with slits defined therethrough, and spacing between the slits is adjustable by moving the distal end of the inner fluid delivery device body with respect to and in contact with a distal end of the interior of the outer fluid delivery device body to adjust the fluid delivered in spray form.

In some aspects, the proximal end is configured to be fluidly coupled with a first lumen of a tubular elongate member configured to deliver the first fluid to the first lumen, and the second lumen of the fluid delivery device is configured to be fluidly coupled with a second lumen of the tubular elongate member fluidly isolated from the first lumen of the tubular elongate member and configured to deliver the second fluid to the second lumen.

In accordance with various principles of the present disclosure, a fluid delivery system includes a fluid delivery device and a tubular elongate member. In some aspects, the fluid delivery device includes a proximal end; a distal end; a first lumen defined through the fluid delivery device to deliver a first fluid to the distal end of the fluid delivery device; and a second lumen defined through the fluid delivery device to deliver a second fluid to the distal end of the fluid delivery device. In some aspects, the tubular elongate member defines a first lumen having a distal end fluidly coupled with the first lumen of the fluid delivery device, and a proximal end fluidly coupleable with a first fluid source; and a second lumen having a distal end fluidly coupled with the second lumen of the fluid delivery device, and a proximal end fluidly coupleable with a second fluid source. In some aspects, the first lumens of the fluid delivery device and the tubular elongate member define a first lumen of the fluid delivery system; the second lumens of the fluid delivery device and the tubular elongate member define a second lumen of the fluid delivery system fluidly isolated from the first lumen of the fluid delivery system; and the first and second lumens of the fluid delivery system end and open to a distal delivery end of the fluid delivery device to deliver the first and second fluids to the distal delivery end and to cause the first and second fluids to be mixed and to be delivered in a spray form.

In some aspects, the first and second lumens of the system are substantially continuous lumens defined by channel walls defined in the fluid delivery device and the tubular elongate member uninterrupted by separate components positioned therein.

In some aspects, the first lumen of the tubular elongate member is fluidly coupled with a fluid source for a viscous first fluid and the second lumen of the tubular elongate member is fluidly coupled with a fluid source for a pressurized second fluid. In some aspects, the distal delivery end of the fluid delivery device is proximally inwardly tapered to direct the pressurized second fluid exiting the second lumen of the fluid delivery system toward the viscous first fluid exiting the first lumen of the fluid delivery system to cause the viscous first fluid to be delivered in a spray form.

In some aspects, the fluid delivery device includes an inner fluid delivery device body defining the first lumen of the fluid delivery device body longitudinally therethrough, and having an exterior surface with one or more grooves therein defining the second lumen of the fluid delivery device body; and the second lumen of the tubular elongate member fits over the exterior of the fluid delivery device body to form the second lumen of the fluid delivery device body with the one or more grooves.

In some aspects, the fluid delivery device includes an outer fluid delivery device defining an interior therein, and an inner fluid delivery device body positioned within the interior of the outer fluid delivery device body; and the outer fluid delivery device body and the inner fluid delivery device body are moveable with respect to each other to adjust the spray form of the fluids exiting therefrom.

In accordance with various principles of the present disclosure, a method of delivering a fluid in spray form includes delivering a first fluid through a first lumen of a fluid delivery device to exit through a distal delivery end of the fluid delivery device; and delivering a second fluid through a second lumen of the fluid delivery device to exit through the distal delivery end of the fluid delivery device and to combine with the first fluid to deliver the first fluid in a spray form.

In some aspects, the method further includes delivering the second fluid as a pressurized fluid causing the first fluid to be delivered from the distal delivery end of the fluid delivery device in a spray form.

In some aspects, the method further includes directing a distal exit end of the second lumen of the fluid delivery device toward a distal exit end of the first lumen of the fluid delivery device to cause the first fluid to be delivered from the distal delivery end of the fluid delivery device in a spray form.

In some aspects, the method further includes delivering the first fluid through the first lumen and the second fluid through the second lumen without encountering separate components with the first and second lumens.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but are intended to be scaled down in relation to, e.g., to fit within a, working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a fluid delivery system formed in accordance with various aspects of the present disclosure.

FIG. 2A illustrates, partially in cross-section, a distal portion of a fluid delivery system formed in accordance with various principles of the present disclosure illustrating an example of an embodiment of a fluid delivery device.

FIG. 2B illustrates a proximal perspective view of a fluid delivery device body of the fluid delivery device of FIG. 2A.

FIG. 2C illustrates an enlarged view of the distal end of the fluid delivery device body illustrated in FIG. 2A and FIG. 2B.

FIG. 3 illustrates, exploded and partially in cross-section, a distal portion of a fluid delivery system formed in accordance with various principles of the present disclosure illustrating an example of an embodiment of a fluid delivery device.

FIG. 4A illustrates, partially exploded and partially in cross-sectional, a distal portion of a fluid delivery system formed in accordance with various principles of the present disclosure, with a change in perspective views on different sides of the break line along the fluid delivery device to illustrate a perspective view of the distal end of an example of an embodiment of a fluid delivery device and a perspective view of the proximal end of the fluid delivery device.

FIG. 4B illustrates an enlarged view of the distal end of the fluid delivery device body illustrated in FIG. 4A.

FIG. 5 illustrates a cross-sectional view through an example of an embodiment of a tubular elongate member with an outer lumen and an inner lumen.

FIG. 6 illustrates a cross-sectional view through an example of an embodiment of a multi-lumen tubular elongate member.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Open surgery (involving cutting open the patient's body to gain access into the patient's body and internal anatomical structures) or laparoscopy (typically involving smaller incisions to gain access into the patient's body and internal anatomical structures) are typical options for performing procedures within a patient's body. Such methods involve longer recovery times than transluminal (e.g., endoscopic) methods which do not involve incisions, but which, instead, typically access the internal anatomy through a natural orifice and advancing equipment, tools, devices, etc., transluminally (through the lumen of a tubular element, such as a catheter, sheath, endoscope, etc.) to the target area/treatment site. The present disclosure describes fluid delivery devices, systems, and methods configured to deliver a material within a patient, such as to deliver the material transluminally. Delivery of materials through lumens of tubular elements small enough to be advanced into and within a body passage of a patient may present various challenges, as noted above. In particular, in some instances, internal procedures involve cutting of tissue (which may bleed), or repair of tissue which may already be bleeding (e.g., a bleeding ulcer). Delivery of a hemostatic material to stop internal bleeding within a patient is particularly challenging, as hemostatic fluid materials tend to be highly viscous. For example, a tremendous amount of force may be required to push a highly viscous fluid through a narrow opening or lumen such as a tubular member sized to be advanced within a patient (e.g., through a body passage of a patient). Traditionally, syringes are used to deliver treatment fluids to a patient. But, the amount of force which may be applied to a syringe may not be sufficient to advance a highly viscous material into a patient for delivery in a desired manner, such as in the form of a spray.

Devices, systems, and methods of the present disclosure are designed to deliver a material to a treatment site within a patient's body. In accordance with various principles of the present disclosure, the material may be a viscous (e.g., highly viscous, such as up to approximately 2000 cP) material such as an adhesive-type or a hemostatic material/agent (e.g., fibrin). The hemostatic material may be selected to create a protective layer that minimizes delayed bleeds, potential perforations, and/or stricture formation within the patient's body. Potential clinical uses include, without limitation, post resection (e.g., endoscopic mucosal resection (EMR), endoscopic submucosal dissection (ESD), polypectomy, etc.), suture line reinforcement, fistula sealing, post Peroral Endoscopic Myotomy (POEM) site closure.

In accordance with various principles of the present disclosure, a fluid delivery device configured to deliver a material within a patient includes a nozzle. In some aspects, the fluid delivery device may alternately be referenced as a fluid nozzle without intent to limit. In some aspects, the nozzle is configured to deliver a viscous material. Alternatively or additionally, in some aspects, the nozzle is configured to deliver a material in a spray form. Alternatively or additionally, in some aspects, the nozzle has a distal end forming a distal delivery end, and a proximal end configured to be fluidly coupled with one or more fluid delivery lumens, such as formed through respective one or more tubular fluid delivery elements. In some aspects, a fluid delivery nozzle formed in accordance with various principles of the present disclosure has at least a first lumen through which a first (e.g., viscous) fluid, such as a hemostatic material/agent, is deliverable, and a second lumen through which a second (e.g., propellant) fluid (e.g., air or carbon dioxide) is deliverable. The nozzle may be formed from a single monolithic nozzle body defining the first and second lumens, or may be formed as a two-part nozzle body defining the first and second lumens. The first and second lumens have distal delivery openings opening adjacent the distal delivery end of the nozzle. In some aspects, the nozzle includes an inner lumen and an outer lumen.

In accordance with various principles of the present disclosure, a nozzle formed in accordance with various principles of the present disclosure, and associated systems and methods, are used to deliver a viscous fluid through a first lumen, and a propellant fluid, such as a pressurized gas, through the second lumen. The materials passed and delivered through the first and second lumens exit the distal delivery end of the nozzle, with the propellant fluid dispersing the viscous fluid in a spray form. In some aspects, the nozzle is adjustable to adjust various parameters, such as the volume, application area, droplet size, etc., of the fluid spray.

In accordance with various further principles of the present disclosure, the fluid delivery system includes a tubular elongate member fluidly coupled between and to the lumens of the fluid delivery device and fluid sources which deliver fluids to the fluid delivery system. The tubular elongate member may include one or more tubular members or shafts defining one or more lumens configured and positioned for fluid coupling with respective lumens of the fluid delivery device.

Various embodiments of fluid delivery devices and systems, and associated methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

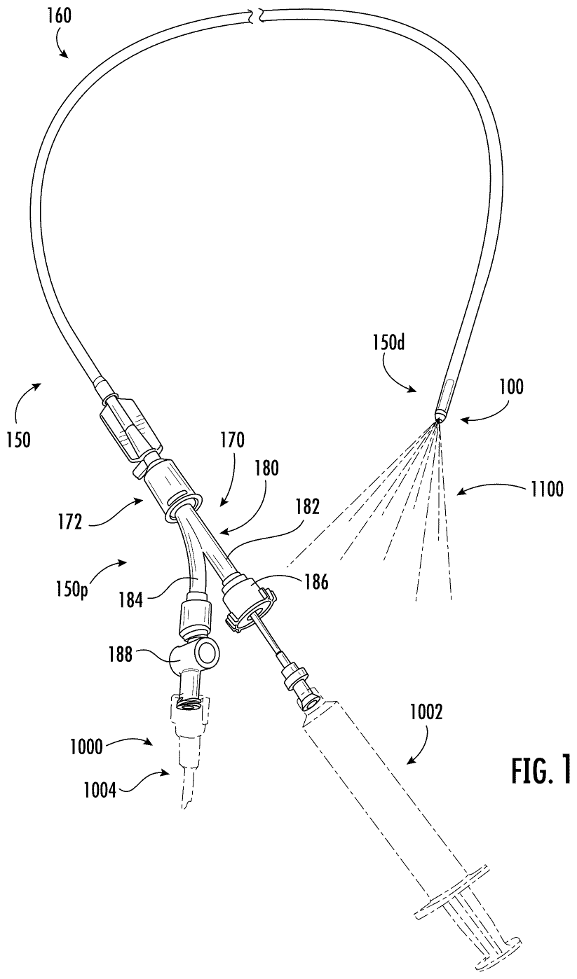

Turning now to the drawings, an example of an embodiment of a fluid delivery device 100 of a fluid delivery system 150, both formed in accordance with various principles of the present disclosure, is illustrated in FIG. 1. As may be appreciated, the fluid delivery device 100 is provided at the distal end 150d of the fluid delivery system 150, with a tubular elongate member 160 extending proximally from the fluid delivery device 100. One or more fluid sources 1000 are fluidly coupled with the tubular elongate member 160 at the proximal end 150p of the fluid delivery system 150 to deliver fluid to the fluid delivery device 100. The fluid sources 1000 may be fluidly coupled with the tubular elongate member 160 via a fluid coupler 170 (described in further detail below). In some aspects, one or more fluids are delivered by one or more respective fluid sources 1000 into one or more respective lumens of the tubular elongate member 160 (e.g., corresponding to each fluid source 1000), as described in further detail below.

The fluid delivery system 150 is configured to deliver a fluid within the body of a patient. For instance, the tubular elongate member 160 may be formed of a material sufficiently flexible to be inserted and navigated through a patient's body, such as through a tortuous natural body passage, to deliver the fluid delivery device 100 to a treatment site at which the fluid is to be delivered (e.g., applied to the treatment site). For instance, the tubular elongate member 160 may be formed of a material such as polytetrafluoroethylene (PTFE); a block copolymer variation of PEBA (polyether block amide), such as PEBAX®; polyether ether ketone (PEEK), etc., such as in the form of a braided catheter, a single lumen extrusion, or other suitable forms known to those of ordinary skill in the art. As illustrated in FIG. 1, the fluid delivery device 100 is configured to deliver a fluid in a spray form 1100. Such delivery form of the fluid may be beneficial to deposit fluid quickly, efficiently, effectively, etc., to the treatment site.

The example of an embodiment of a fluid delivery system 150 illustrated in FIG. 1 is configured to deliver a first fluid through a first lumen, and a second fluid through a second lumen, the lumens meeting at the distal end 150d of the fluid delivery system 150 to mix the fluids to create a desired fluid in a spray form 1100. In accordance with various further principles of the present disclosure, the first fluid may be a viscous fluid and the second fluid may be a less viscous fluid (e.g., a gas such as air or CO₂) which may function as a propellant for the first fluid, such that upon mixing the first and second fluids at the distal end 150d of the fluid delivery system 150, the second fluid disperses and delivers the first fluid in spray form. In accordance with various principles of the present disclosure, the fluid delivery device 100 is configured to facilitate mixing of the first and second fluids to result in delivery of the first fluid in a spray form, as will now be described.

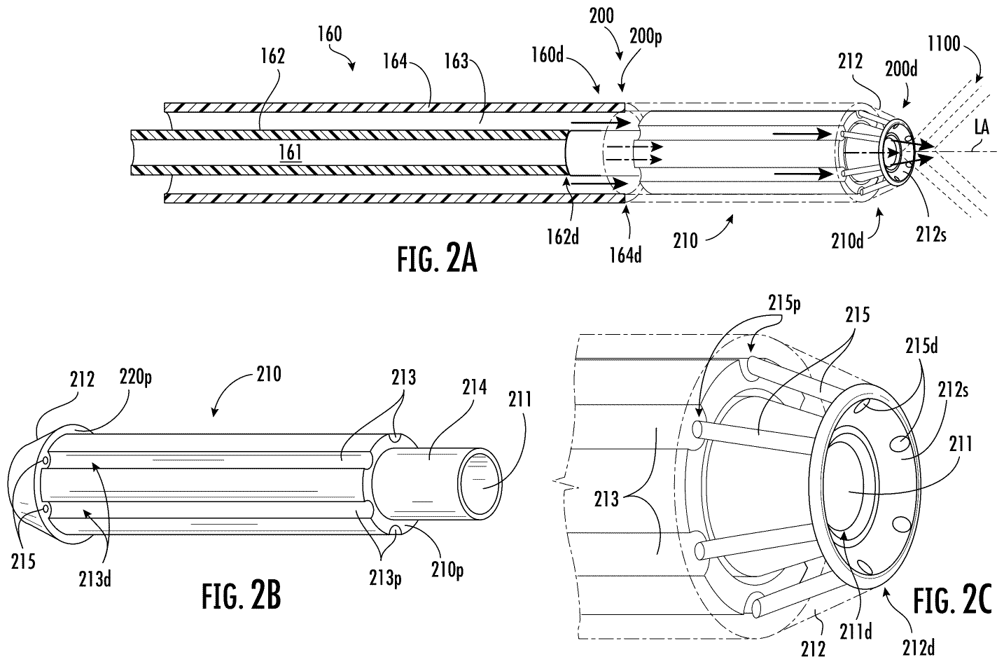

Turning to FIG. 2A, FIG. 2B, and FIG. 2C, a distal portion of a fluid delivery system 150 formed in accordance with various principles of the present disclosure is shown to illustrate an example of an embodiment of a fluid delivery device 200. In accordance with various principles of the present disclosure, the fluid delivery device 200 includes a fluid delivery device body 210 (which may alternately be referenced as a nozzle body 210) defining a first lumen 211 through which a first fluid is delivered to a treatment site, and a second lumen 213 through which a second fluid is delivered to a treatment site. In the illustrated example of an embodiment, the first lumen 211 is an internal lumen defined generally longitudinally through the fluid delivery device body 210, such as axially through the center of the fluid delivery device body 210, such as illustrated in FIG. 2B. In some aspects, the second lumen 213 comprises one or more lumens, such as illustrated in FIG. 2B and FIG. 2C, functioning together to deliver a second fluid. More particularly, in the example of an embodiment of a fluid delivery device body 210 illustrated in FIG. 2A, FIG. 2B, and FIG. 2C, the second lumen 213 includes one or more external grooves or channels on the exterior surface of the fluid delivery device body 210. The distal end 210d of the fluid delivery device body 210 tapers to form a distal delivery end 212 of the fluid delivery device 200 with one or more channels 215 therethrough having proximal end 215p in fluid communication with the one or more second lumens 213, and distal open ends 215d exiting the distal end 200d of the fluid delivery device 200 to deliver the second fluid to a treatment site, such as by mixing with the first fluid exiting the distal open end 211d of the first lumen 211 to form a fluid in a spray form 1100. In some aspects, the distal end 212d of the distal delivery end 212 may be proximally inwardly tapered to facilitate mixing of the second fluid exiting the second lumen 213 with the first fluid exiting the first lumen 211. It will be appreciated that the example of an embodiment of a fluid delivery device body 210 illustrated in FIG. 2A, FIG. 2B, and FIG. 2C is a single, monolithic member. However, other embodiments include two or more separately-formed members together forming a fluid delivery device body, as described in further detail below.

The proximal end 200p of the fluid delivery device 200 is fluidly coupled with the distal end 160d of the tubular elongate member 160 to facilitate delivery of a first fluid and a second fluid, respectively, through the lumens 211, 213 of the fluid delivery device body 210 and out the distal end 200d of the fluid delivery device 200 for delivery of a fluid in a spray form 1100. The distal portion of the fluid delivery system 150 is illustrated in FIG. 2A in an exploded and partial cross-sectional view to illustrate a first lumen 161 and a second lumen 163 defined through the tubular elongate member 160. More particularly, the illustrated example of an embodiment of a tubular elongate member 160 is formed of a first tubular member 162 defining the first lumen 161, and a second tubular member 164 defining the second lumen 163. In the illustrated example of an embodiment, the first tubular member 162 is an inner tubular member defining an inner lumen, and the second tubular member 164 is an outer tubular member defining an outer lumen. The first tubular member 162 may be generally substantially concentrically disposed within the second tubular member 164 to define the first lumen 161 generally concentrically extending within and through the second lumen 163. However, other configurations are within the scope of the present disclosure, such as to fluidly couple with corresponding lumens of an associated fluid delivery device body. Thus, in the example of an embodiment illustrated in FIG. 2A, FIG. 2B, and FIG. 2C, the first lumen 161 of the tubular elongate member 160 and the first lumen 211 of the fluid delivery device body 210 are configured and positioned to be fluidly coupled with each other. Similarly, the second lumen 163 of the tubular elongate member 160 and the second lumen 213 of the fluid delivery device body 210 are configured and positioned to be fluidly coupled with each other. As may be appreciated, the tubular elongate member 160 is fluidly coupled with the proximal end 200p of the fluid delivery device 200 (e.g., along the illustrated directional arrows) to deliver fluid to the distal end 200d of the fluid delivery device 200.

As may be appreciated with reference to FIG. 2A, the distal end 164d of the second tubular member 164 extends distally beyond the distal end 162d of the first tubular member 162 a sufficient distance to be extended over the exterior surface of the fluid delivery device body 210 along which the second lumens 213 are formed. As may further be appreciated, the portion of the fluid delivery device body 210 along which the second lumens 213 are defined has an outer diameter smaller than the outer diameter of the proximal end 220p of the distal delivery end 212. The second tubular member 164 may thereby be extended over the exterior of the fluid delivery device body 210 defining the second lumens 213 until the distal end 164d of the second tubular member 164 abuts the proximal end 212p (e.g., a circumferential surface or shoulder defined along the proximal end 212p) of the distal delivery end 212. As such, although the second lumens 213 defined on the exterior of the fluid delivery device body 210 may be open channels, the interior of the second tubular member 164 encloses such open channels to form, with the second lumens 213, a lumen through which the second fluid is delivered to the distal end 200d of the fluid delivery device 200. As may be further appreciated with reference to FIG. 2B and FIG. 2C, the distal ends 213d of the second lumens 213 are in fluid communication with channels 215 defining lumens through the distal delivery end 212 of the fluid delivery device body 210.

As may be appreciated with reference to FIG. 2A and FIG. 2B, the first tubular member 162 may be fluidly coupled with a proximal coupling extension 214 extending proximally beyond the proximal ends 213p of the second lumens 213 and the exterior surface of the fluid delivery device body 210 along which the second lumens 213 are defined. The distal end 162d of the first tubular member 162 may be inserted into the first lumen 211 to form a preferably fluid-tight connection to fluidly separate the lumen formed by the first lumens 161, 211 and the lumen formed by the second lumens 163, 213 to isolate the first and second fluids from each other as they are delivered to the distal end 200d of the fluid delivery device 200. Alternatively, the distal end 162d of the first tubular member 162 may be extended over the proximal coupling extension 214 and up to a proximal shoulder or surface 210p of the fluid delivery device body 210 radially-inward of the proximal ends 213p of the second lumens 213 to fluidly communicate the first lumens 161, 211 with a preferably fluid tight connection to fluidly isolate the first and second fluids.

The fluid communication and fluid tight connection between the tubular elongate member 160 and the fluid delivery device 200 allows fluids to be delivered from a fluid source, and through the tubular elongate member 160 and the fluid delivery device 200, to be delivered to a treatment site. More particularly, a first fluid may be delivered (from a first fluid source, as described in further detail below) through the first lumens 161, 211 and out the distal end 211d of the first lumen 211. And, a second fluid may be delivered (from a second fluid source, as described in further detail below) through the second lumens 163, 213 and out the distal ends 215d of the channels 215. The first and second fluids exiting the respective distal ends 211d, 213d, 215d of the lumens 211, 213, and channels 215 may mix or combine at the distal end 200d of the fluid delivery device 200.

In accordance with various principles of the present disclosure, a second fluid in the form of a pressurized fluid, such as a pressurized gas, may disperse the first fluid to deliver a fluid in a spray form 1100 to a treatment site. In some aspects, such as illustrated in FIG. 2C, and noted above, the distal end 212d of the distal delivery end 212 of the fluid delivery device 200 has a proximally inwardly tapered surface 212s directed inwardly into the distal delivery end 212. The distal ends 215d of the channels 215 through the distal delivery end 212 thus are directly radially inwardly to direct the second fluid towards the first fluid as the first fluid exits the first lumen 211. Such configuration may better facilitate mixing of the first fluid and the second fluid than if the second fluid exited the lumens 315 axially. Alternatively or additionally, such proximally tapered surface 212s widens distally and therefore may facilitate dispersion of the first and second fluids in a wider spray form 1100 than if the surface were cylindrical about the longitudinal axis LA of the fluid delivery device 200. In some aspects, the proximally tapered surface 212s has a substantially 90° taper (i.e., the surface tapered approximately 45° with respect to the longitudinal axis LA of the fluid delivery device 200).

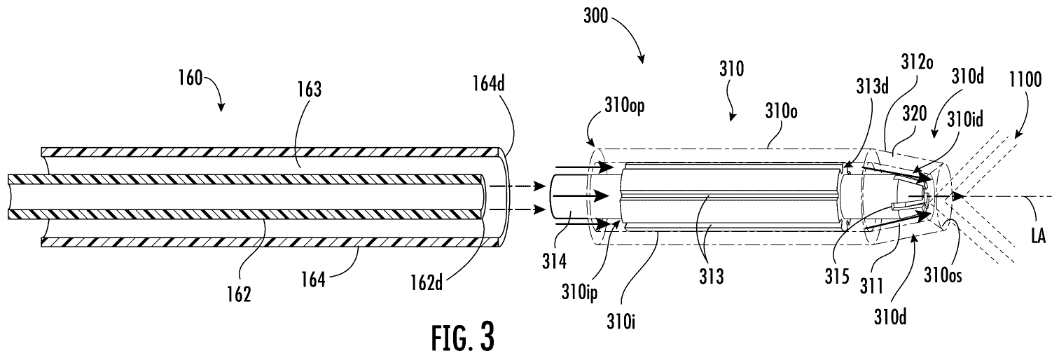

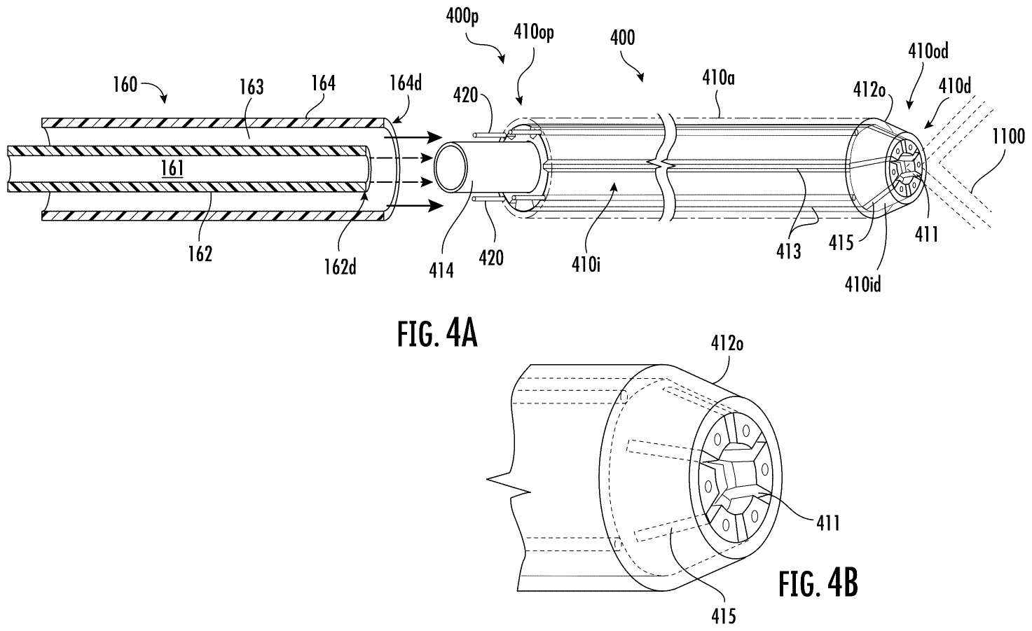

Instead of a fluid delivery device 200 with a fluid delivery body 210 defining lumens for both the first fluid and the second fluid delivered thereto by the tubular elongate member 160, such as illustrated in FIG. 2A, FIG. 2B, and FIG. 2C, a fluid delivery device may have a two-part fluid delivery body. For instance, the fluid delivery device may be formed of an inner fluid delivery device body fitted within the interior (e.g., a cavity, channel, etc.) of a generally hollow outer fluid delivery device body. The inner and outer fluid delivery device bodies cooperate to form first and second lumens for respective first and second fluids. Examples of embodiments of distal portions of a fluid delivery system 150 with fluid delivery devices 300, 400 formed of at least two separately formed members are illustrated in FIG. 3, and FIG. 4A and FIG. 4B, which will now be described.

Turning now to FIG. 3, a distal portion of a fluid delivery system 150 with another example of an embodiment of a fluid delivery device 300 is illustrated. As may be appreciated, the illustrated fluid delivery device 300 has a fluid delivery device body 310 made up of an inner fluid delivery device body 310i (which may alternately be referenced as an inner fluid nozzle, inner shaft, etc., without intent to limit) and an outer fluid delivery device body 310o (illustrated in phantom so that the inner fluid delivery device body 310i may be viewed, and which may alternately be referenced as an air nozzle top cap without intent to limit). As in FIG. 2A, the distal portion of the fluid delivery system 150 is illustrated in FIG. 3 in an exploded and partial cross-sectional view to illustrate a first lumen 161 and a second lumen 163 defined through the tubular elongate member 160, similar to those illustrated in FIG. 2A. Accordingly, a more detailed description of the elements of the tubular elongate member 160 is omitted for the sake of brevity, reference being made to the above descriptions of the tubular elongate member 160 illustrated in FIG. 2A as generally applying to the tubular elongate member 160 illustrated in FIG. 3, with the same reference numbers being used.

Like the fluid delivery device body 210 of the fluid delivery device 200 illustrated in FIG. 2A, FIG. 2B, and FIG. 2C, the inner fluid delivery device body 310i of the fluid delivery device 300 illustrated in FIG. 3 defines a first lumen 311 therethrough, and one or more second lumens 313 along an exterior surface thereof. However, instead of the second tubular member 164 enclosing the second lumens 313, the interior of the outer fluid delivery device body 310o (within which the inner fluid delivery device body 310i is positioned) encloses the otherwise open channels forming the second lumens 313 in the exterior surface of the inner fluid delivery device body 310i. The second tubular member 164 is thus inserted over the proximal coupling extension 314 extending proximally from the proximal end 310ip of the inner fluid delivery device body 310i and up to a proximal shoulder or surface 310op of the outer fluid delivery device body 310o, such as to form a fluid tight fit. The first tubular member 162 may either be inserted within or over the proximal coupling extension 314 to form a fluid tight fit (e.g., friction fit) with respect to the inner fluid delivery device body 310i. The fluid-tight fits may be friction fits, or friction fits enhanced by an adhesive (e.g., UV glue).

In the illustrated example of an embodiment, the distal ends 313d of the second lumens 313 end proximal to a distally tapered (e.g., conical) distal delivery end 312o of the outer fluid delivery device body 310o. The second fluid exits the second lumen 313 into the interior space defined by the distally tapered distal delivery end 312o and combines with the first fluid exiting the first lumen 311 within the distally tapered distal delivery end 312o. In some aspects, the distal end 310id of the inner fluid delivery device body 310i is distally tapered (e.g., conical), optionally at the same degree of taper as the distally tapered distal delivery end 312o of the outer fluid delivery device body 310o. In the example of an embodiment illustrated in FIG. 3, one or more slits 315 are formed in the distal end 310id of the inner fluid delivery device body 310i, and the first fluid exits the first lumen 311 through the slits 315 to combine/mix with the second fluid within the distally tapered distal delivery end 312o of the outer fluid delivery device body 310o. In some aspects, the slits 315 allow a viscous first fluid delivered through the outer fluid delivery device body 310o to have quick contact with the second fluid delivered through the radial spaces between the slits 315 and the outer fluid delivery device body 310o. Alternatively or additionally, the distal end 310od of the outer fluid delivery device body 310o may have a proximally inwardly tapered surface 310os directed inwardly into the distal end 310od. Such proximally tapered surface 310os widens distally and therefore may facilitate dispersion of the first and second fluids in a wider spray form 1100 than if the surface were cylindrical about the longitudinal axis LA of the fluid delivery device 300.

As may be appreciated, as with the example of an embodiment of a fluid delivery device 200 illustrated in FIG. 2A, FIG. 2B, and FIG. 2C, the example of an embodiment of a fluid delivery device 300 illustrated in FIG. 3 is in fluid communication with the tubular elongate member 160, with a fluid tight connection therebetween, to allows fluids to be delivered from a fluid source, and through the tubular elongate member 160 and the fluid delivery device 300 to be delivered to a treatment site. In some aspects, the tapered distal end 310id of the inner fluid delivery device body 310i may be inserted within the outer fluid delivery device body 310o sufficiently distally to contact the inner surface of the distally tapered distal delivery end 312o of the outer fluid delivery device body 310o. Once the first tubular member 162 and the second tubular member 164 are coupled, respectively, to the inner fluid delivery device body 310i and the outer fluid delivery device body 310o, the relative positions of the inner fluid delivery device body 310i and the outer fluid delivery device body 310o may be set (e.g., no longer movable with respect to each other, such as fixed with respect to each other).

As may be appreciated, the example of an embodiment of a fluid delivery device 200 illustrated in FIG. 2A, FIG. 2B, and FIG. 2C may have a larger outer diameter than the outer diameter of the example of an embodiment of a fluid delivery device 300 illustrated in FIG. 3. When intended to be advanced through a working channel of an endoscope, the outer diameters of the fluid delivery devices 200, 300 are typically less than about 2.7 mm (a typical inner diameter of a working channel of an endoscope), such as approximately 2.65 mm. Whereas the fluid delivery device 200 may be able to apply fluid in a spray form 1100 to a treatment site of about 45-50 mm in diameter, the fluid delivery device 300 may apply fluid in a spray form 1100 to a smaller treatment site area. And, both fluid delivery devices 200, 300 generally apply fluid in a spray form 1100 with a generally fixed spray angle.

In accordance with various principles of the present disclosure, a two-part fluid delivery device (e.g., similar to the fluid delivery device 300 of FIG. 3) may be configured to allow adjustability of the spray angle at which the fluid is delivered in spray form 1100 to a treatment site T. In the example of an embodiment of a distal portion of a fluid delivery system 150 illustrated in FIG. 4A and FIG. 4B, the fluid delivery system 150 includes a fluid delivery device 400 with a fluid delivery device body 410 formed as a two-part part body similar to the fluid delivery device body 310 of the fluid delivery device 300. However, the inner fluid delivery device body 410i and the outer fluid delivery device body 410o (illustrated in phantom so that the inner fluid delivery device body 410i may be viewed) forming the fluid delivery device body 410 of the fluid delivery device 400 illustrated in FIG. 4A and FIG. 4B are movable with respect to each other to allow the fluid in a spray form 1100 to be adjusted (e.g., velocity, volume, spray area, etc.). Other than features and elements allowing adjustability of the relative positions of the inner fluid delivery device body 410i and the outer fluid delivery device body 410o, the various features, elements, components, etc. of the fluid delivery device 400 are substantially the same as the fluid delivery device 300. Accordingly, for the sake of brevity, and without intent to limit, reference is made to the above detailed descriptions of features, elements, components, etc., of the fluid delivery device 300 as being generally applicable to the fluid delivery device 400, unless otherwise indicated herein. For the sake of convenience, the same reference numerals indicating features, elements, components, etc., of the fluid delivery device 300 are used, but increased by 100, to indicate features, elements, components, etc., of the fluid delivery device 400. Likewise, the fluid coupling of the tubular elongate member 160 with the proximal end 400p of the fluid delivery device 400 is substantially the same as described above with reference to the fluid delivery device 300 illustrated in FIG. 3. Accordingly, for the sake of brevity, and without intent to limit, reference is made to the above detailed descriptions of the fluid connections between the tubular elongate member 160 and the fluid delivery device 300 as applying mutatis mutandis to the tubular elongate member 160 and the fluid delivery device 400, with the same reference numerals increased by 100 being used for features, elements, components, etc., of the fluid delivery device 400.

Turning now to FIG. 4A, the illustrated example of an embodiment of a fluid delivery device 400 includes an inner fluid delivery device body 410i within an outer fluid delivery device body 410o, similar to the arrangement of the example of an embodiment of a fluid delivery device 300 illustrated in FIG. 3. However, as may be appreciated with reference to the proximal perspective end view of the fluid delivery device 400, one or more pull wires 420 extend proximally from the proximal end 410op of the outer fluid delivery device body 410o. In some embodiments, the pull wires 420 extend through channels within the wall thickness of the outer fluid delivery device body 410o. However, other configurations (such as extending along the interior or exterior of the outer fluid delivery device body 410o, e.g., through open channels/grooves therein) are within the scope and spirit of the present disclosure. Pulling proximally on the one or more pull wires 420 moves the outer fluid delivery device body 410o proximally with respect to the inner fluid delivery device body 410i (which is fixed in position). As noted above, the fluid delivery device 400 has various features, elements, components, etc., in common with the fluid delivery device 300, including a distally tapered distal delivery end 412o of the outer fluid delivery device body 410o, and a distally tapered distal end 410id of the inner fluid delivery device body 410i. And, like the inner fluid delivery device body 310i of the fluid delivery device 300, the inner fluid delivery device body 410i of the fluid delivery device 400 includes one or more slits 415. As may be appreciated with reference to FIG. 4B, the distally tapered distal end 410id of the inner fluid delivery device body 410i and the slits 415 therethrough are configured similar to a collet. As such, relative movement between the distal end 410od of the outer fluid delivery device body 410o and the distal end 410id of the inner fluid delivery device body 410i alter the shape of the distally tapered distal end 410id of the inner fluid delivery device body 410i and the spacing/width of the slits 415. Such modifications alter the various properties (velocity, volume, spray area, etc.) of the fluid delivered in a spray form 1100 as may be appreciated by those of ordinary skill in the art. As may further be appreciated by those of ordinary skill in the art, various further adjustments, such as to the volumetric flow rate, the flow velocity, the pressure, etc., to the flow of the second liquid may be made to adjust the manner, degree, extent, etc., to which the second liquid disperses the first liquid into a spray form 1000.

As may be appreciated, the lumens of the various examples of embodiments of fluid delivery devices 200, 300, 400 described above are free of valves or other components positioned therein which may affect fluid flow through the lumens. Instead, the lumens provided generally unobstructed, generally continuous pathways/passages for fluid flow from the one or more fluid sources of the fluid delivery system 150 to the distal end 150d of the fluid delivery system 150 at which the one or more fluids are delivered by the fluid delivery system 150 to a patient. Likewise, the lumens 161, 163 through the tubular elongate member 160 are free of valves or other components positioned therein which may affect fluid flow therethrough. As such, the fluid delivery system 150 described herein has a fluid delivery device with a generally cylindrical fluid delivery device body defining, with a tubular elongate member, lumens of the system which themselves are configured to mix one or more fluids to deliver a fluid, such as a viscous fluid, in a spray form, without further devices and/or components. As may further be appreciated, the various examples of embodiments of fluid delivery devices 200, 300, 400 described above do not require rotational or angular alignment or a particular relative rotational or angular orientation with respect to each other, thereby facilitating assembly and use.

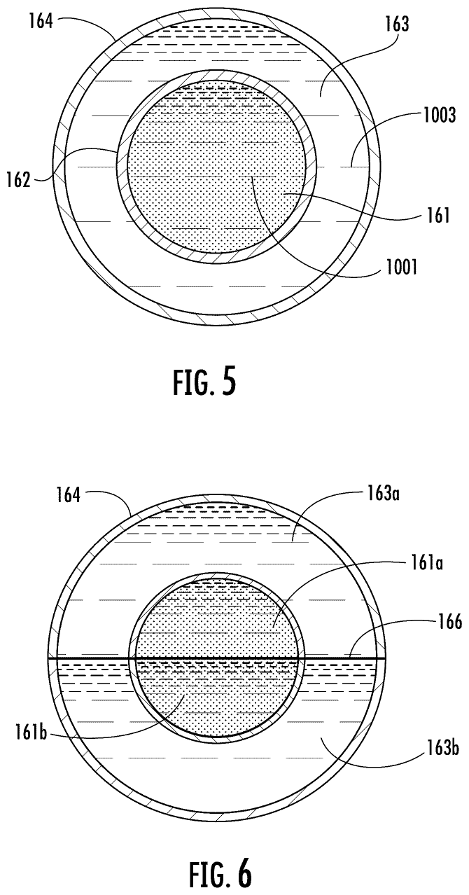

Nonetheless, fluid isolation of the various lumens through which different fluids are delivered to the distal end 150d of the fluid delivery system 150 fluid coupler 170 must be maintained. As schematically illustrated in FIG. 5, showing a cross-section through the tubular elongate member 160 at an unspecified location, the first lumen 161 is fluidly isolated from the second lumen 163 to fluidly isolate the first fluid 1001 (within the first lumen 161) from the second fluid 1003 (within the second lumen 163). In some aspects, instead of delivering just two fluids, the tubular elongate member 160 may be configured to deliver more than two fluids fluidly isolated from one another. For instance, in the example of an embodiment illustrated in FIG. 6, a dividing wall 166 separates the first tubular member 162 into a first half-lumen 161a and a second half-lumen 161b, and separates the second tubular member 164 into a first half-lumen 163a and a second half-lumen 163b, fluidly isolating the fluids in each fluidly isolated lumen. It will be appreciated that the present disclosure is not limited by the precise configuration of the lumens, and other configurations are within the scope and spirit of the present disclosure.

Returning to FIG. 1, as noted above, a fluid coupler 170 is configured to fluidly couple the fluid delivery system 150 with one or more fluid sources 1000. In order to maintain the first fluid and the second fluid separate, the lumens through which the first fluid and the second fluid are delivered to the distal end 150d of the fluid delivery system 150 are maintained fluidly isolated from one another. In accordance with various principles of the present disclosure, the fluid coupler 170 is configured to fluidly couple the first lumen of the fluid delivery system 150 (formed of fluidly coupled first lumens within the tubular elongate member 160 and the fluid delivery device 100, 200, 300, 400) with a first fluid source 1010, and to fluidly couple the second lumen of the fluid delivery system 150 (formed of fluidly coupled second lumens within the tubular elongate member 160 and the fluid delivery device 100, 200, 300, 400) with a second fluid source 1020 in a manner which maintains the fluid isolation of the first and second fluids. In the example of an embodiment of a fluid coupler 170 illustrated in FIG. 1, such fluid isolation is achieved by the fluid coupler 170 including a Luer lock 172 with concentric lumens therein respectively fluidly coupled with the first lumen 161 and the second lumen 163 of the tubular elongate member 160 in a manner which fluidly isolates the lumens 161, 163. A Y-connector 180 (which, in some aspects, may be considered a part of the fluid coupler 170) fluidly couples separate fluid sources with the separate lumens of the Luer lock 172. More particularly, the Y-connector 180 includes a first branch 182 and a second branch 184. As illustrated in FIG. 1, the first branch 182 is fluidly coupled between and with a first fluid source 1002 and the Luer lock 172 (specifically, with the inner lumen of the Luer lock 172, which is not illustrated, but is well understood by those of ordinary skill in the art with reference to commercially available Lucr locks). As also illustrated in FIG. 1, the second branch 184 is fluidly coupled between and with a second fluid source 1004 and the Luer lock 172 (specifically, with the outer lumen of the Luer lock 172, which is not illustrated, but is well understood by those of ordinary skill in the art with reference to commercially available Luer locks). In the illustrated example of an embodiment, the first fluid source 1002 includes a syringe filled with the first fluid, such as a viscous fluid, such as a hemostatic agent, and the second fluid source 1004 includes an inlet tube coupled with a fluid source which may pressurize the second fluid (e.g., an air inflation pump, a miniature compressor for delivering a pressurized fluid such as air, a prefilled CO₂ cylinder handle, etc.). In some aspects, the first branch 182 of the Y-connector 180 includes a seal 186, such as known to those of ordinary skill in the art, to control/prevent any leakage of fluid from the first fluid source 1002 into the Y-connector 180, and/or to prevent mixing of fluids within the fluid coupler 170. In some aspects, the second branch 184 of the Y-connector 180 includes a control valve 188 which (optionally with the second fluid source 1004) may facilitate regulation of the flow of the second fluid from the second fluid source 1004 into the fluid coupler 170. A seal, such as known to those of ordinary skill in the art, may also be provided in the second branch 184 and/or within the Luer lock 172 to prevent mixing/leaking of the second fluid with the first fluid. As discussed above, the second fluid is pumped into the fluid delivery system 150 to reach the distal end 150d thereof and exit the fluid delivery system 150 with the first viscous fluid. The pressurized second fluid pushes the more viscous first fluid out of the fluid delivery system 150 into a fluid in a spray form 1100.

The proximal end 150p of the fluid delivery system 150 (e.g., including various components of the fluid coupler 170) may be configured to be operated by a human operator (e.g., physician and/or technician and/or individual assisting the physician) or an automated (e.g., robotic) operating system such as in a manner known to those of ordinary skill in the art. As may be appreciated, the proximal end 150p may be configured as an ergonomic interface with an intuitive user interface facilitating quick attachment to an endoscope, thereby involving minimal setup time for use, and improved usability compared with prior art systems. For instance, the fluid delivery system 150 may be provided pre-assembled and ready for use by a medical professional with no additional activities or steps required (except connection to fluid sources). Moreover, a fluid delivery system 150 formed in accordance with various principles of the present disclosure allows for delivery of a viscous fluid in a spray form not previously achievable by existing spray device or systems.

It should be apparent to those of ordinary skill in the art that variations can be applied to the disclosed devices, systems, and/or methods, and/or to the sequence of steps of the method described herein without departing from the concept, spirit, and scope of the disclosure.

The foregoing discussion has broad application and has been presented for purposes of illustration and description and is not intended to limit the disclosure to the form or forms disclosed herein. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A fluid delivery device comprising:
a proximal end;
a distal end;
a first lumen defined through said fluid delivery device to deliver a first fluid to said distal end of said fluid delivery device; and
a second lumen defined through said fluid delivery device to deliver a second fluid to said distal end of said fluid delivery device;
wherein:
said first lumen and second lumen end and open to a distal delivery end of said fluid delivery device to deliver the first and second fluids to said distal delivery end and to cause said first and second fluids to be mixed and to be delivered in a spray form.

2. The fluid delivery device of claim 1, wherein said first lumen and second lumen are concentrically arranged with respect to each other.

3. The fluid delivery device of claim 2, wherein said proximal end is configured to be fluidly coupled with a tubular elongate member defining a first lumen concentrically positioned within a second lumen, said first lumen of said fluid delivery device is configured and positioned to be fluidly coupled with the first lumen of the tubular elongate member, and said second lumen of said fluid delivery device is configured and positioned to be fluidly coupled with the second lumen of the tubular elongate member.

4. The fluid delivery device of claim 1, wherein said fluid delivery device comprises an inner fluid delivery device body defining said first lumen longitudinally therethrough and having an exterior surface with one or more grooves therein defining said second lumen.

5. The fluid delivery device of claim 4, wherein said distal delivery end of said fluid delivery device defines one or more channels therethrough, said one or more grooves defining said second lumen fluidly coupled with respective ones of said one or more channels to define said second lumen.

6. The fluid delivery device of claim 5, wherein said one or more channels end and open to a proximally inwardly tapered distal end of said distal delivery end to direct the second fluid toward the first fluid as the first fluid exits the first lumen to deliver the first and second fluids in a spray form.

7. The fluid delivery device of claim 4, further comprising an outer fluid delivery device body defining an interior, wherein said inner fluid delivery device body is positioned within the interior of said outer fluid delivery device body, the interior of said outer fluid delivery device body defining said second lumen with said one or more grooves in the exterior surface of said inner fluid delivery device body.

8. The fluid delivery device of claim 7, wherein said inner fluid delivery device body and said outer fluid delivery device body are movable with respect to each other to adjust the flow of fluid through said distal delivery end of said fluid delivery device.

9. The fluid delivery device of claim 8, wherein said inner fluid delivery device body has a distal end with slits defined therethrough, and spacing between the slits is adjustable by moving said distal end of said inner fluid delivery device body with respect to and in contact with a distal end of the interior of said outer fluid delivery device body to adjust the fluid delivered in spray form.

10. The fluid delivery device of claim 1, wherein said proximal end is configured to be fluidly coupled with a first lumen of a tubular elongate member configured to deliver the first fluid to said first lumen, and said second lumen of said fluid delivery device is configured to be fluidly coupled with a second lumen of the tubular elongate member fluidly isolated from the first lumen of the tubular elongate member and configured to deliver the second fluid to said second lumen.

11. A fluid delivery system comprising:
a fluid deliver device comprising:
a proximal end;
a distal end;
a first lumen defined through said fluid delivery device to deliver a first fluid to said distal end of said fluid delivery device; and
a second lumen defined through said fluid delivery device to deliver a second fluid to said distal end of said fluid delivery device; and
a tubular elongate member defining:
a first lumen having a distal end fluidly coupled with said first lumen of said fluid delivery device, and a proximal end fluidly coupleable with a first fluid source; and
a second lumen having a distal end fluidly coupled with said second lumen of said fluid delivery device, and a proximal end fluidly coupleable with a second fluid source;
wherein:
said first lumens of said fluid delivery device and said tubular elongate member define a first lumen of said fluid delivery system;
said second lumens of said fluid delivery device and said tubular elongate member define a second lumen of said fluid delivery system fluidly isolated from said first lumen of said fluid delivery system; and
said first and second lumens of said fluid delivery system end and open to a distal delivery end of said fluid delivery device to deliver the first and second fluids to said distal delivery end and to cause said first and second fluids to be mixed and to be delivered in a spray form.

12. The fluid delivery system of claim 11, wherein said first and second lumens of said system are substantially continuous lumens defined by channel walls defined in said fluid delivery device and said tubular elongate member uninterrupted by separate components positioned therein.

13. The fluid delivery system of claim 11, wherein said first lumen of said tubular elongate member is fluidly coupled with a fluid source for a viscous first fluid and said second lumen of said tubular elongate member is fluidly coupled with a fluid source for a pressurized second fluid.

14. The fluid delivery system of claim 13, wherein the distal delivery end of said fluid delivery device is proximally inwardly tapered to direct the pressurized second fluid exiting said second lumen of said fluid delivery system toward the viscous first fluid exiting said first lumen of said fluid delivery system to cause said viscous first fluid to be delivered in a spray form.

15. The fluid delivery system of claim 11, wherein:
said fluid delivery device comprises an inner fluid delivery device body defining said first lumen of said fluid delivery device body longitudinally therethrough, and having an exterior surface with one or more grooves therein defining said second lumen of said fluid delivery device body; and
said second lumen of said tubular elongate member fits over the exterior of said fluid delivery device body to form said second lumen of said fluid delivery device body with said one or more grooves.

16. The fluid delivery system of claim 11, wherein:
said fluid delivery device comprises an outer fluid delivery device defining an interior therein, and an inner fluid delivery device body positioned within the interior of said outer fluid delivery device body; and
said outer fluid delivery device body and said inner fluid delivery device body are moveable with respect to each other to adjust the spray form of the fluids exiting therefrom.

17. A method of delivering a fluid in spray form, said method comprising:
delivering a first fluid through a first lumen of a fluid delivery device to exit through a distal delivery end of the fluid delivery device; and
delivering a second fluid through a second lumen of the fluid delivery device to exit through the distal delivery end of the fluid delivery device and to combine with the first fluid to deliver the first fluid in a spray form.

18. The method of claim 17, further comprising delivering the second fluid as a pressurized fluid causing the first fluid to be delivered from the distal delivery end of the fluid delivery device in a spray form.

19. The method of claim 17, further comprising directing a distal exit end of the second lumen of the fluid delivery device toward a distal exit end of the first lumen of the fluid delivery device to cause the first fluid to be delivered from the distal delivery end of the fluid delivery device in a spray form.

20. The method of claim 17. comprising delivering the first fluid through the first lumen and the second fluid through the second lumen without encountering separate components with the first and second lumens.
